(19) United States
(12) Patent Application Publication
(10) Pub. No.: US 2007/0005103 A1
(43) Pub. Date: Jan. 4, 2007

(54) Emboli capturing device having a netted outer surface

(75) Inventor: Darin G. Schaeffer, Bloomington, IN (US)

Correspondence Address: BRINKS HOFER GILSON & LIONE/CHICAGO/COOK, PO BOX 10395, CHICAGO, IL, 60610, US

(73) Assignee: COOK INCORPORATED, BLOOMINGTON, IN (US)

(21) Appl. No.: 11/479,219

(22) Filed: Jun. 30, 2006

Related U.S. Application Data
(60) Provisional application No. 60/695,510, filed on Jun. 30, 2005.

(51) Int. Cl. A61M 29/00

(52) U.S. Cl. 606/200

(57) ABSTRACT

An emboli capture device with a netted wall for capturing emboli during treatment during of a stenotic lesion in a body vessel is disclosed. The device comprises an expandable stent having a partially-expanded state, a fully-expanded state and a closed state. The expandable stent includes a continuous filament configured about a longitudinal axis to define an outer surface of the stent. The device further comprises a filter portion attached about the outer surface of the stent for capturing emboli. The filter portion is placed between the outer surface and the body vessel when the stent is in the partially-expanded state for capturing emboli.

### CROSS-REFERENCE TO RELATED APPLICATION

This application claims the benefit of U.S. Provisional Application No. 60/695,510, filed on Jun. 30, 2005, entitled “Emboli Capturing Device Having A Netted Outer Surface,” the entire contents of which are incorporated herein by reference.

### BACKGROUND OF THE INVENTION

The present invention relates to medical devices. Particularly, the present invention relates to an emboli capturing device with a netted outer surface for capturing emboli during treatment of a stenotic lesion in a body vessel.

Embolic protection to capture emboli within the vasculature is a growing concern in the medical industry. Currently, there are a number of approaches for embolic protection to prevent emboli from traveling within the vasculature to create an undesirable embolism, e.g., pulmonary embolism. For example, vena cava filters are more commonly being used for trapping emboli in the vena cava filter to prevent pulmonary embolism. Also, anti-platelet agents and anticoagulants may be used to breakdown blood clots. Moreover, snares and baskets (e.g., stone retrieval baskets) are more commonly used for retrieving urinary calculi. Additionally, occlusion coils are commonly used to occlude aneurysms and accumulate thrombi in a body vessel.

Treatments for a stenotic lesion provide a potential in releasing blood clots and other thrombi plaque in the vasculature of the patient. One example is the treatment for a carotid artery stenosis. Generally, carotid artery stenosis is the narrowing of the carotid arteries, the main arteries in the neck that supply blood to the brain. Carotid artery stenosis (also called carotid artery disease) is a relatively high risk factor for ischemic stroke. The narrowing is usually caused by plaque build-up in the carotid artery. Plaque forms when cholesterol, fat and other substances form in the inner lining of an artery. This formation process is called atherosclerosis.

Depending on the degree of stenosis and the patient's overall condition, carotid artery stenosis has been treated with surgery. The procedure (with its inherent risks) is called carotid endarectomy, which removes the plaque from the arterial walls. Carotid endarectomy has proven to benefit patients with arteries substantially narrowed, e.g., by about 70% or more. For people with less narrowed arteries, e.g., less than about 50%, an anti-clotting drug may be prescribed to reduce the risk of ischemic stroke. Examples of these drugs are anti-platelet agents and anticoagulants.

Carotid angioplasty is a more recently developed treatment for carotid artery stenosis. This treatment uses balloons and/or stents to open a narrowed artery. Carotid angioplasty is a procedure that can be performed via a standard percutaneous transfemoral approach with the patient anesthetized using light intravenous sedation. At the stenosis area, an angioplasty balloon is delivered to pre-dilate the stenosis in preparation for stent placement. The balloon is then removed and exchanged via catheter for a stent delivery device. Once in position, a stent is deployed across the stenotic area. If needed, an additional balloon can be placed inside the deployed stent for post-dilation to make sure the struts of the stent are pressed firmly against the inner surface of the vessel wall.

During the stenosis procedure however, there is a risk of such blood clots and thrombi being undesirably released into the blood flow within the vasculature. Embolic or distal protection devices have been implemented to capture emboli from a stenotic lesion undergoing angioplasty. However, many current emboli capture devices restrict flow when deployed within the vasculature of the patient. Moreover, many emboli capture devices are relatively difficult to collapse and retrieve after the need for such device in the vasculature passes.

Thus, there is a need to provide a device and method for distally capturing and trapping emboli within a body lumen during a stenosis procedure.

### BRIEF SUMMARY OF THE INVENTION

In one embodiment, the present invention provides an embolic protection and emboli capture device with a netted wall for capturing emboli during treatment of a stenotic lesion in a body vessel. The device comprises an expandable stent having a partially-expanded state, a fully-expanded state and a closed state. The expandable stent is configured about a longitudinal axis to define an outer surface of the stent. The device further comprises a filter portion attached about the stent for capturing and trapping emboli. The filter portion is placed against the body vessel when the stent is in the partially-expanded and fully-expanded states for capturing emboli.

In another embodiment, the present invention provides an emboli capture assembly for capturing emboli during treatment of a stenotic lesion in a body vessel. In this embodiment, the assembly comprises a balloon catheter having a tubular body portion and an expandable balloon attached to and in fluid communication with the tubular body portion for angioplasty at the stenotic lesion. The expandable balloon has distal and proximal portions. The assembly further includes the emboli capture device. In this embodiment, the expandable stent of the device includes a filament having struts connected together by bends configured about the longitudinal axis to define an outer surface of the stent. The device is coaxially disposed through the balloon catheter during treatment of the stenotic lesion in the body vessel.

In another example, present invention provides a method for capturing emboli during treatment of a stenotic lesion in a body vessel. The method comprises percutaneously introducing the emboli capture assembly. The method further comprises deploying the device in its partially-expanded state downstream from the stenotic lesion to capture emboli during treatment of the stenotic lesion and engaging emboli during treatment of the stenotic lesion. The method further comprises deploying the device in the fully-expanded state to capture the emboli between the device and the body vessel.

Further objects, features, and advantages of the present invention will become apparent from consideration of the following description and the appended claims when taken in connection with the accompanying drawings.

### BRIEF DESCRIPTION OF THE DRAWINGS

FIG. 1 is an environmental view of an emboli capture device in a partially-deployed state in accordance with one embodiment of the present invention;

FIG. 2 is a perspective side view of the emboli capture device in FIG. 1;

FIG. 3 is an environmental view of the device in a fully-deployed state;

FIG. 4 is a side perspective view of the device in the fully-deployed state;

FIG. 5 is a side view of the device in a collapsed state within a delivery member;

FIG. 6 is a plan view of the device in accordance with one embodiment of the present invention;

FIG. 7a is a side view of an emboli capture assembly for capturing emboli during treatment of a stenotic lesion in a body vessel in accordance with one embodiment of the present invention;

FIG. 7b is an exploded view of the assembly in FIG. 7a;

FIG. 8 is a flow chart of one method for capturing emboli during treatment of a stenotic lesion in a body vessel; and

FIG. 9 is a side view of an emboli capture assembly in accordance with another embodiment of the present invention.

### DETAILED DESCRIPTION OF THE INVENTION

The present invention generally provides an emboli capture device for capturing emboli during treatment of a stenotic lesion in a body vessel. Embodiments of the present invention provide a device that captures undesirably released emboli and traps the emboli from traveling further downstream of the vasculature of a patient. The device further has a reduced cross-sectional profile for delivery of the device prior to pre-dilitation of the stenotic lesion, and a relatively simple manner of trapping the emboli therein. In one embodiment, the device includes an expandable stent having a closed state, a partially-expanded state, and a fully-expanded state. In the closed state, the device is allowed to have a reduced cross-sectional profile for delivery through a stenosed area. In the partially-expanded state, the device captures emboli from an upstream angioplasty treatment. In the fully-expanded state, emboli are trapped between the outer surface of the device and the body vessel.

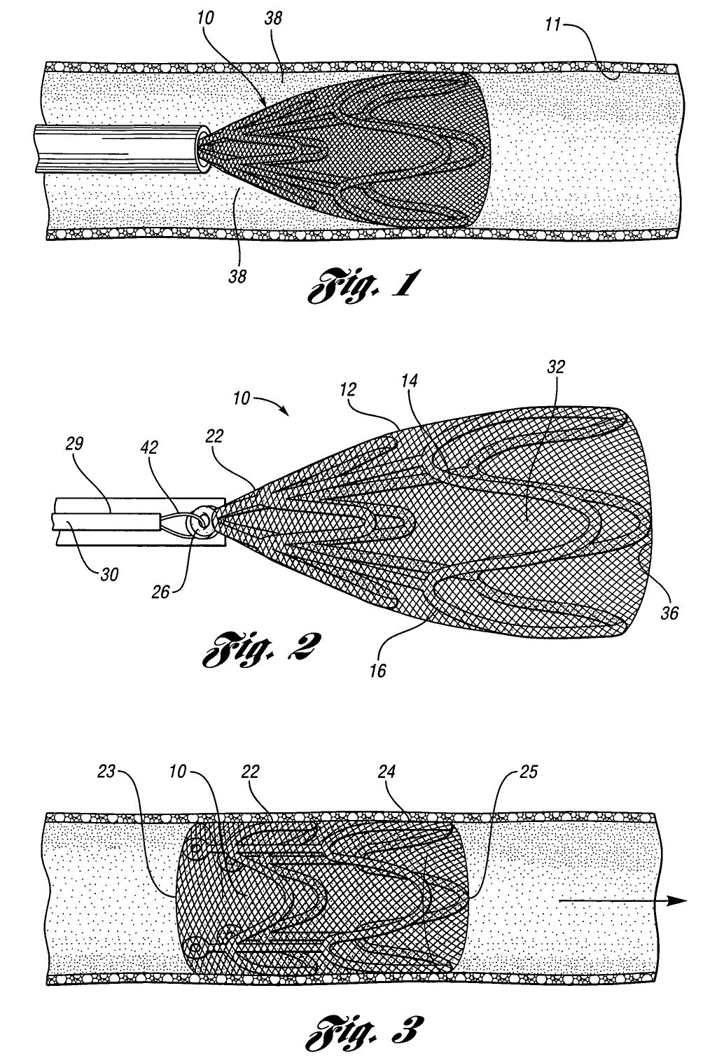

FIG. 1 illustrates an emboli capture device 10 for capturing and trapping emboli during treatment of a stenotic lesion in a body vessel 11 in accordance with one embodiment of the present invention. As depicted in FIGS. 1 and 2, the emboli capture device 10 comprises an expandable stent 12 including a continuous filament 14 having a plurality of struts 16 connected together. The expandable stent 12 may be formed in any suitable configuration or manner to provide the continuous filament 14 or a plurality of filaments 14 having the plurality of struts 16 connected together. For example, the continuous filament 14 may be a single continuous filament 14 having struts 16 connected together by arcuate bends axially extending therealong. The expandable stent 12 that is configured to expand radially upon deployment. As shown, the filament 14 is axially configured about a longitudinal axis A to define an outer surface 20 of the stent 12. Of course, the filament 14 may be configured in any other suitable manner, e.g., spirally. The outer surface 20 is preferably a cylindrical surface. Preferably, the expandable stent 12 is configured to self-expand to the fully-expanded state.

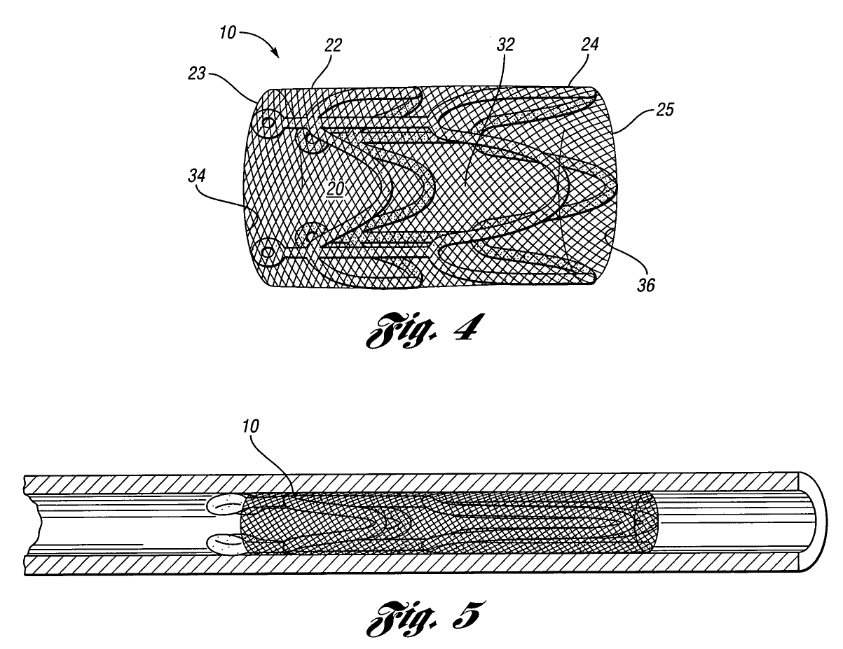

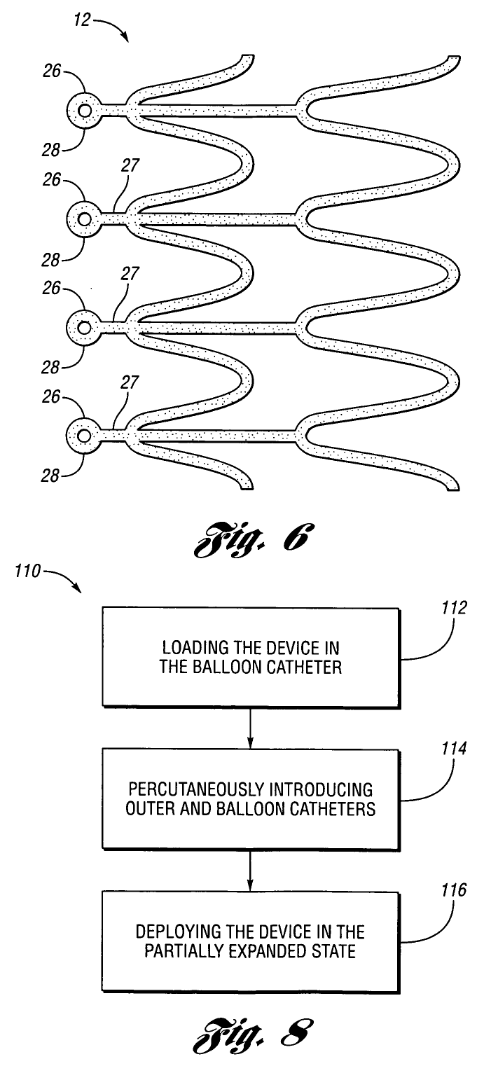

In this embodiment, the expandable stent 12 is formed to have a closed state (FIG. 5), a partially-expanded state (FIGS. 1 and 2), and a fully-expanded state (FIGS. 3 and 4). In the closed state, the device 10 is provided a reduced cross-sectional profile for delivery through a stenosed area. In the partially-expanded state, the device 10 is configured to capture or wedge emboli from an upstream angioplasty treatment. In the fully-expanded state, the device 10 is formed to trap emboli between the outer surface 20 of the device 10 and the body vessel while maintaining blood flow therethrough.

In this embodiment, the expandable stent 12 has a proximal portion 22 including a proximal end 23 and a distal portion 24 including a distal end 25. As depicted in FIGS. 2-4, in the fully-expanded state, each of the proximal end 23 and the distal end 25 is open to allow for blood to flow therethrough. Preferably, the proximal end 23 may comprise deployment members 26 that, when cooperating together, allow for controlled deployment of the device 10. As shown in FIGS. 2, 4, and 6, each of the deployment members 26 has a neck 27 and a ring 28 integral with the neck 27. The members cooperate together with a delivery system for relatively precise and accurate deployment of the device 10 prior to treatment of a stenotic lesion in a body vessel.

FIG. 2 illustrates the emboli capture device 10 in the partially-expanded state in which the device 10 is partially deployed from a tubular member, e.g., a catheter 29. As shown, the expandable stent 12 is partially deployed from the catheter 29 for capturing or filtering emboli during treatment of a stenotic lesion in a body vessel. The device 10 may be associated with a delivery mechanism 30 that cooperates with the deployment members 26 of the expandable stent 12. For example, the delivery mechanism 30 may include a maneuverable grasping loop 42 for grasping the neck 27 of each of the deployment members 26 together. In the partially-expanded state, the deployment members 26 cooperate together to allow the proximal portion 22 of the expandable stent 12 to remain grasped and undeployed for capturing emboli during angioplasty. In this example, the grasping loop 42 may then be manipulated to release the deployment members 26 in the fully-expanded state for trapping emboli that are captured.

FIGS. 3 and 4 depict the device 10 in its fully-expanded state to which the stent 12 is configured to self-expand for trapping the emboli between the device 10 and the body vessel wall on which it engages. The device 10 preferably takes on a cylindrical shape in its fully-expanded state. As shown, each of the proximal end 23 and the distal end 25 is an open end. This is to allow blood to flow therethrough similar to a typical expandable angioplasty stent 12. As mentioned, the device 10 is biased to self-expand to its fully-expanded state. The self-expanding configuration provides an outward radial force to engage the outer surface with the body vessel.

FIGS. 2 and 4 illustrate the device 10 further comprising a filter portion 32 attached about the outer surface 20 of the expandable stent 12 for trapping emboli. Preferably, the filter portion 32 has a proximal edge 34 and a distal edge 36. The proximal edge 34 of the filter portion 32 is attached to the proximal portion 22, preferably at the proximal end 23, of the expandable stent 12 and extends distally therefrom. The distal edge 36 is attached to the distal portion 24, preferably at the distal end 25, of the expandable stent 12. Thus, the filter portion 32 preferably attaches entirely about the outer surface 20 of the expandable stent 12.

FIG. 5 illustrates the emboli capture device 10 in the closed state. As shown, the device 10 is loaded in a tubular member, e.g., a catheter 29, providing a reduced cross-sectional profile for delivery through a stenosed area. As discussed above, the delivery mechanism having the grasping loop may be used to axially advance the device loaded within a catheter. However, it is understood that any other suitable delivery mechanism may be used without falling beyond the scope or spirit of the present invention. For example, a pusher or push wire may be used to distally advance the device within a catheter to a desired deployment location in the vasculature of a patient. As the device is deployed from the distal end of the catheter, the deployed portion of the device is axially biased outwardly to self-expand and engage the vessel wall in which the device is inserted (in the partially-expanded state), thereby capturing released emboli. Upon release of the grasping loop from the rings, the catheter may be retracted relative to the delivery mechanism 30 to fully deploy the device 10 against the vessel wall (in the fully-deployed state), thereby trapping emboli.

The expandable stent 12 may be comprised of any suitable material such as a superelastic material, stainless steel wire, cobalt-chromium-nickel-molybdenum-iron alloy, or cobalt-chrome alloy. It is understood that the expandable stent 12 may be formed of any other suitable material that will result in a self-opening or self-expanding expandable stent 12, such as shape memory alloys. Shape memory alloys have the desirable property of becoming rigid, that is, returning to a remembered state, when heated above a transition temperature. A shape memory alloy suitable for the present invention is Ni—Ti available under the more commonly known name Nitinol. When this material is heated above the transition temperature, the material undergoes a phase transformation from martensite to austenic, such that material returns to its remembered state. The transition temperature is dependent on the relative proportions of the alloying elements Ni and Ti and the optional inclusion of alloying additives.

In one embodiment, the expandable stent 12 is made from Nitinol with a transition temperature that is slightly below normal body temperature of humans, which is about 98.6° F. Thus, when the expandable stent 12 is deployed in a body vessel and exposed to normal body temperature, the alloy of the expandable stent 12 will transform to austenite, that is, the remembered state, which for one embodiment of the present invention is the expanded state when the expandable stent 12 is deployed in the body vessel. To remove the expandable stent 12, the expandable stent 12 is cooled to transform the material to martensite which is more ductile than austenite, making the expandable stent 12 more malleable. As such, the expandable stent 12 can be more easily collapsed and pulled into a lumen of a catheter 29 for removal.

In another embodiment, the expandable stent 12 is made from Nitinol with a transition temperature that is above normal body temperature of humans, which is about 98.6° F. Thus, when the expandable stent 12 is deployed in a body vessel and exposed to normal body temperature, the expandable stent 12 is in the martensitic state so that the expandable stent 12 is sufficiently ductile to bend or form into a desired shape, which for the present invention is an expanded configuration. To remove the expandable stent 12, the expandable stent 12 is heated to transform the alloy to austenite so that the expandable stent 12 becomes rigid and returns to a remembered state, which for the expandable stent 12 in the closed state.

The filter portion 32 may be comprised of any suitable material to be used for capturing and trapping emboli from the stenotic lesion during treatment thereof. In one embodiment, the filter portion 32 is made of connective tissue material for capturing emboli. In this embodiment, the connective tissue comprises extracellular matrix (ECM). As known, ECM is a complex structural entity surrounding and supporting cells that are found within mammalian tissues. More specifically, ECM comprises structural proteins (e.g., collagen and elastin), specialized protein (e.g., fibrillin, fibronectin, and laminin), and proteoglycans, a protein core to which are attached are long chains of repeating disaccharide units termed of glycosaminoglycans.

Most preferably, the extracellular matrix is comprised of small intestinal submucosa (SIS). As known, SIS is a resorbable, acellular, naturally occurring tissue matrix composed of ECM proteins and various growth factors. SIS is derived from the porcine jejunum and functions as a remodeling bioscaffold for tissue repair. SIS has characteristics of an ideal tissue engineered biomaterial and can act as a bioscaffold for remodeling of many body tissues including skin, body wall, musculoskeletal structure, urinary bladder, and also supports new blood vessel growth. In many aspects, SIS is used to induce site-specific remodeling of both organs and tissues depending on the site of implantation. In theory, host cells are stimulated to proliferate and differentiate into site-specific connective tissue structures, which have been shown to completely replace the SIS material in time.

In other embodiments, the filter portion 32 may also be made of a mesh/net cloth, nylon, bio-polymeric material, polytetrafluoroethylene (PTFE), e.g., Teflon™, porous polyurethane biomaterial (e.g., Thoralon™), or woven mixtures thereof without falling beyond the scope or spirit of the present invention.

Upon deployment from the catheter 29, the distal portion 24 of the expandable stent 12 self-expands radially, thereby pressing a part of the filter portion 32 against the wall to capture emboli undesirably released during angioplasty. As FIG. 1 depicts the device 10 in the partially-expanded state of the device 10, the filter portion 32 partially engages the vessel wall and is placed between the expandable stent 12 and the body vessel. As a result, the distal portion 24 of the expandable stent 12 is deployed and the proximal portion 22 is undeployed as the delivery mechanism 30 maintains engagement with the rings 28 and maneuverability of the device 10. This advantageously forms a receiving area 38 for capturing emboli released during angioplasty.

Upon release from the delivery mechanism 30 and deployment from the catheter 29, the proximal portion 22 of the expandable stent 12 expands radially, thereby fully pressing the filter portion 32 against the vessel wall to trap the emboli that were captured. As FIG. 3 depicts the device 10 in the fully-expanded state, the outer surface 20 of the filter portion 32 is placed in substantially full engagement with the vessel wall and is held between the expandable stent 12 and the wall. As a result, the device 10 is fully deployed and the outer surface 20 fully presses the vessel wall. This covers the emboli that were captured during angioplasty and traps the emboli between the filter portion 32 and the vessel wall while allowing blood to flow therethrough. In time, the SIS material of the filter portion 32 will begin to differentiate into site-specific connective tissue structures as mentioned above.

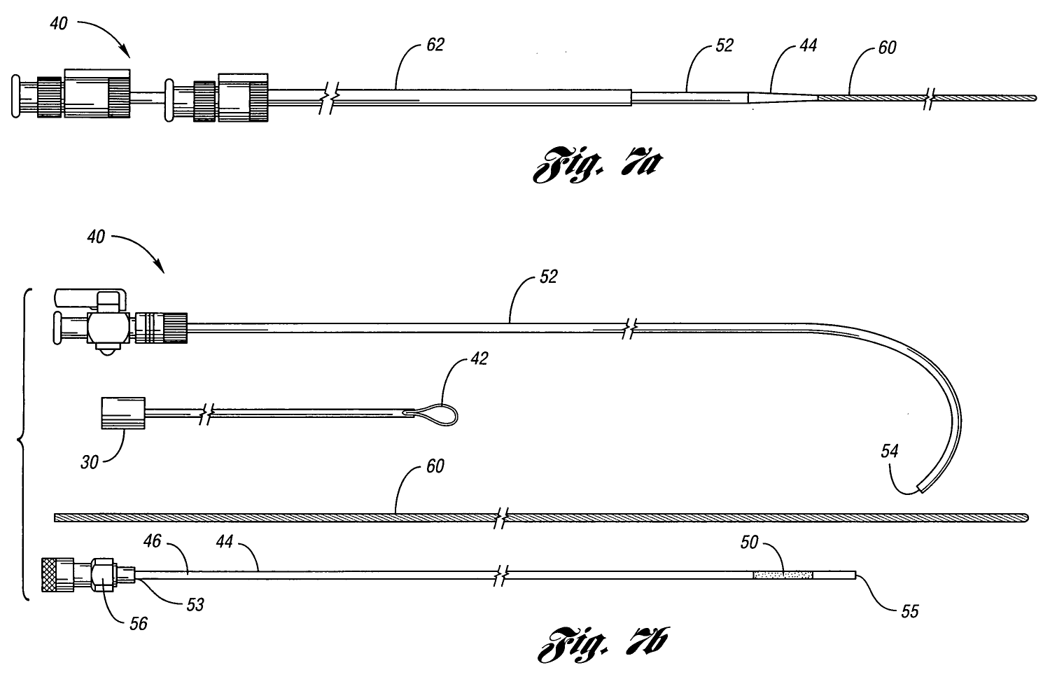

FIGS. 7a and 7b illustrate an emboli capture assembly 40 for capturing emboli during treatment of a stenotic lesion in a body vessel in accordance with another embodiment of the present invention. As shown, the assembly 40 comprises a delivery mechanism 30. In this embodiment, the delivery mechanism 30 is an elongated member having a grasping loop 42 extending through the end of the mechanism and maneuverable relative thereto. As mentioned above, in cooperation with the deployment member of the stent 12, the grasping loop 42 may be manipulated to advance and maintain the device 10 in its partially-expanded state. The grasping loop 42 may then be moved, e.g., loosened, to release the deployment member thereby allowing the stent 12 to fully expand.

FIGS. 7a and 7b further depict the assembly 40 comprising a balloon catheter 44 having a tubular body 46 and an expandable balloon 50 attached to and in fluid communication with the tubular body 46 for angioplasty at a stenotic lesion. In this embodiment, the assembly 40 comprises the emboli capture device 10 mentioned above. The tubular body 46 is preferably made of soft flexible material such as silicon or any other suitable material. In this embodiment, the balloon catheter 44 includes an outer lumen and an inner lumen. The outer lumen is in fluid communication with the balloon for inflating and deflating the balloon. The inner lumen is formed therethrough for percutaneous guidance through the body vessel.

As shown, the assembly 40 further includes an outer catheter 52 having a distal end 54 through which the balloon catheter 44 is disposed for deployment in the body vessel. The outer catheter 52 is preferably made of a soft, flexible material such as silicon or any other suitable material. Generally, the outer catheter 52 further has a proximal end 56 and a plastic adaptor or hub to receive the emboli capture device 10 and balloon catheter 44 to be advanced therethrough. The size of the outer catheter 52 is based on the size of the body vessel in which it percutaneously inserts, and the size of the balloon catheter 44.

As shown, the assembly 40 may also include a wire guide 60 configured to be percutaneously inserted within the vasculature to guide the outer catheter 52 to a location adjacent a stenotic lesion. The wire guide 60 provides the outer catheter 52 (and balloon catheter 44) a path during insertion within the body vessel. The size of the wire guide 60 is based on the inside diameter of the outer catheter 52.

In one embodiment, the balloon catheter 44 has a proximal fluid hub 48 in fluid communication with the balloon via the outer lumen for fluid to be passed therethrough for inflation and deflation of the balloon during treatment of the stenotic lesion.

As shown, the emboli capture device 10 is coaxially disposed through the inner lumen of the balloon catheter 44 prior to treatment of the stenotic lesion in the body vessel. The distal protection device 10 is guided through the inner lumen preferably from the hub and distally beyond the balloon of the balloon catheter 44, exiting from the distal end 25 of the inner or balloon catheter 44 to a location within the vasculature downstream of the stenotic lesion.

In this embodiment, the apparatus further includes a polytetrafluoroethylene (PTFE) introducer sheath 62 for percutaneously introducing the wire guide 60 and the outer catheter 52 in a body vessel. Of course, any other suitable material may be used without falling beyond the scope or spirit of the present invention. The introducer sheath 62 may have any suitable size, e.g., between about three-french to eight-french. The introducer serves to allow the inner and balloon catheters 44 to be percutaneously inserted to a desired location in the body vessel. The introducer sheath 62 receives the outer catheter 52 and provides stability to the outer catheter 52 at a desired location of the body vessel. For example, the introducer sheath 62 is held stationary within a common visceral artery, and adds stability to the outer catheter 52, as the outer catheter 52 is advanced through the introducer sheath 62 to a dilatation area in the vasculature.

When the distal end 54 of the outer catheter 52 is at a location downstream of the dilatation area in the body vessel, the balloon catheter 44 is inserted therethrough to the dilatation area. The device 10 is then loaded at the proximal end 53 of the balloon catheter 44 and is advanced through the inner lumen thereof for deployment through its distal end 55. In this embodiment, the proximal stem is used to mechanically advance or push the device 10 through the catheter 29.

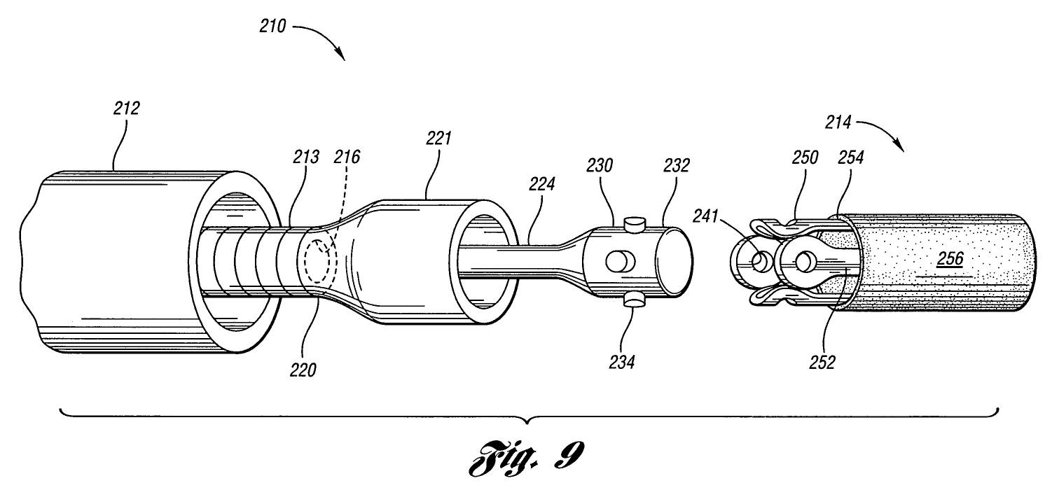

FIG. 8 depicts a flow chart of one method 110 for capturing and trapping emboli during treatment of a stenotic lesion in a body vessel. In this example, the assembly and device discussed above may be used. The wire guide is percutaneously inserted in the vasculature of the patient at a deployment location adjacent a stenotic lesion to provide a path that subsequently guides the outer catheter and balloon catheter to the deployment location. The device is attached to the delivery mechanism (discussed above) and loaded in box 112 in the inner lumen of the balloon catheter which, in turn, is disposed through the outer catheter for insertion into the vasculature.

The method 110 further comprises percutaneously introducing in box 114 the outer catheter and balloon catheter in the vasculature. The outer catheter is advanced through the vasculature and is crossed over the stenotic lesion. The method 110 further includes deploying the device in box 116 in its partially-expanded state downstream from the stenotic lesion to capture emboli during treatment of the stenotic lesion. This may be accomplished by partially deploying the device through the distal ends of the balloon catheter and the outer catheter while the delivery mechanism maintains engagement with the device to place the device in its partially-expanded state. In its partially-expanded state (discussed above), the device has a receiving area defined by the space between the outer surface thereof and the vessel wall, placing the device in a position to capture emboli.

The treatment of the stenotic lesion, e.g., angioplasty, is then performed on the patient. As mentioned, the device captures emboli released upstream during angioplasty. When treatment is completed and the stenosis condition has passed, the device is ready to be placed in its fully-expanded state. The method further includes deploying the device in its fully-expanded state to trap emboli captured during treatment of the stenotic lesion. This may be accomplished by fully deploying the device from the balloon catheter and the outer catheter, and then disengaging the delivery mechanism from the deployment member of the stent. In one example, the grasping loop may be loosened to disengage the delivery mechanism from the device, thereby placing the device in its fully-expanded state. In its fully-expanded state, the device self-expands radially and the outer surface of the device covers the captured emboli, trapping the emboli between the outer surface and the vessel wall.

FIG. 9 illustrates an emboli capture assembly 210 for capturing emboli during treatment of a stenotic lesion in a body vessel in accordance with yet another embodiment of the present invention. As shown, the assembly 210 comprises a catheter 212, e.g., micro-catheter, through which a wire guide 213 is disposed for deployment of an emboli capture device 214. As shown, the wire guide 213 has a lumen 216 (shown in phantom) formed therethrough. In this embodiment, the wire guide 213 has a distal portion 220 that extends to a cap end 221. Preferably, the wire guide 213 is cannular-shaped to allow a pusher 224 to be disposed through the lumen 216 of the wire guide 213. Preferably, the pusher 224 has a distal end 230 comprising a coupler 232. In this embodiment, the coupler 232 includes radial pegs 234 formed about the coupler 232 about which the device 214 attaches for delivery as described in greater detail below.

It is understood that the device 214 has components similar to the components of the device 10 mentioned above. For example, the device 214 comprises an expandable stent 250, a filament 252, struts 254, and a filter portion 256 similar to the expandable stent 12, the filament 14, struts 16, and the filter portion 32 of the device 10.

As shown in FIG. 9, the device 214 has a proximal portion 240 including struts 254 having holes 241 formed therethrough. Each of the proximal holes 241 mates with one of the radial pegs 234 formed about the coupler 232. Thus, in the closed state, the device 214 mates with the coupler 232 and is held about the pusher 224 within the cap end 221 of the wire guide 213. The pusher 224 is configured to be pulled back inside the wire guide 213. In turn, the wire guide 213 configured to be pulled back inside the catheter 212 for delivery of the device 214.

While the present invention has been described in terms of preferred embodiments, it will be understood, of course, that the invention is not limited thereto since modifications may be made to those skilled in the art, particularly in light of the foregoing teachings.

1. An emboli capture device with a netted wall for capturing emboli during treatment of a stenotic lesion in a body vessel, the device comprising:
an expandable stent having a partially-expanded state, a fully-expanded state and a closed state, the expandable stent formed about a longitudinal axis to define an outer surface of the stent; and
a filter portion attached about the outer surface of the stent for capturing emboli, the filter portion being placed between the outer surface and the body vessel when the stent is in the partially-expanded state for capturing emboli.

2. The device of claim 1 wherein the outer surface has a cylindrical shape in the fully-expanded state.

3. The device of claim 1 wherein the stent includes a continuous filament spirally configured about the longitudinal axis to define the outer surface of the stent.

4. The device of claim 1 wherein the filter portion is made of at least one of an extracellular matrix, a mesh cloth, nylon, a bio-polymeric material, polytetrafluoroethylene, porous polyurethane biomaterial, and woven mixtures thereof.

5. The device of claim 4 wherein the extracellular matrix is small intestine submucosa.

6. The device of claim 1 wherein the expandable stent includes proximal and distal portions.

7. The device of claim 6 wherein the partially-expanded state is defined by partial deployment of the stent, whereat the distal portion is deployed and the proximal portion is undeployed.

8. The device of claim 6 wherein the fully-expanded state is defined by full deployment of the stent, whereat the proximal and distal portions are deployed to trap emboli between the outer surface and the body vessel.

9. The device of claim 1 wherein the expandable stent is made of superelastic material.

10. An emboli capture assembly for capturing emboli during treatment of a stenotic lesion in a body vessel, the assembly comprising:
a balloon catheter having a tubular body portion and an expandable balloon attached to and in fluid communication with the tubular body portion for angioplasty at the stenotic lesion, the expandable balloon having distal and proximal portions; and
an emboli capture device coaxially disposed through the balloon catheter during treatment of the stenotic lesion in the body vessel, the device comprising:
an expandable stent having a partially-expanded state, a fully-expanded state and a closed state, the expandable stent including a filament having struts connected together by bends configured about a longitudinal axis to define an outer surface of the stent; and
a filter portion attached about the outer surface of the stent for capturing and trapping emboli between the cylindrical surface and the body vessel when the stent is in the partially-expanded state.

11. The assembly of claim 10 wherein the balloon catheter includes an outer lumen and an inner lumen, the outer lumen being in fluid communication with the balloon for inflating and deflating the balloon, the inner lumen formed therethrough for percutaneous guidance through the body vessel.

12. The assembly of claim 10 further comprising:
an outer catheter having a distal end through which the balloon catheter is disposed for deployment in the body vessel;
a wire guide configured to be disposed through the inner lumen of the balloon catheter for percutaneous guidance through the body vessel; and
an introducer sheath through which the outer catheter is inserted for percutaneous insertion to the body vessel.

13. The assembly of claim 10 wherein the outer catheter further includes a proximal end, the proximal end having a hub in fluid communication with the balloon for fluid to be passed therethrough for inflation and deflation of the balloon during treatment of the stenotic lesion.

14. The assembly of claim 10 wherein the bends are arcuate bends.

15. The assembly of claim 10 wherein the filament is spirally configured about the longitudinal axis to define the cylindrical surface of the stent.

16. The assembly of claim 10 wherein the filter portion is made of at least one of an extracellular matrix, a mesh cloth, nylon, a bio-polymeric material, polytetrafluoroethylene, a porous polyurethane biomaterial, and woven mixtures thereof.

17. The assembly of claim 10 wherein the extracellular matrix is small intestine submucosa.

18. The assembly of claim 10 wherein the expandable stent includes proximal and distal portions.

19. The assembly of claim 18 wherein the partially-expanded state is defined by partial deployment of the stent, whereat the distal portion is deployed and the proximal portion is undeployed.

20. An emboli capture assembly for capturing emboli during treatment of a stenotic lesion in a body vessel, the assembly comprising:
a catheter having a tubular body portion;
a wire guide having a lumen formed therethrough and being disposed through the catheter, the wire guide having a distal portion extending to a cap end portion;
an elongate pusher member disposed through the lumen of the wire guide, the elongate pusher member comprising a distal end, the distal end having a coupler formed thereon, the coupler including pegs formed radially about the coupler; and
an emboli capture device coaxially disposed through the catheter during treatment of the stenotic lesion in the body vessel, the device comprising:
an expandable stent having a partially-expanded state, a fully-expanded state and a closed state, the expandable stent including a filament comprising struts connected together by bends configured about a longitudinal axis to define an outer surface of the stent, the filament comprising a proximal end having holes formed therethrough, each of the hole being configured to mate with a peg to attach about the coupler in the closed state; and
a filter portion attached about the outer surface of the stent for capturing and trapping emboli between the cylindrical surface and the body vessel when the stent is in the partially-expanded state.

21. A method for capturing emboli during treatment of a stenotic lesion in a body vessel, the method comprising:
percutaneously introducing an emboli capture assembly having a balloon catheter and an emboli capture device disposed coaxially within the balloon catheter, the device comprising:
an expandable stent having a partially-expanded state, a fully-expanded state and a closed state, the expandable stent including a continuous filament configured about a longitudinal axis to define an outer surface of the stent;
a filter portion attached about the outer surface of the stent for capturing emboli, the filter portion being placed between the outer surface and the body vessel when the stent is in the partially-expanded state for capturing emboli.
deploying the device in the partially-expanded state downstream from the stenotic lesion to capture emboli during treatment of the stenotic lesion;
engaging emboli during treatment of the stenotic lesion; and
deploying the device in the fully-expanded state to capture the emboli between the device and the body vessel.
